(19) United States
(12) Patent Application Publication
(10) Pub. No.: US 2008/0014580 A1
(43) Pub. Date: Jan. 17, 2008

(54) Detection of biological molecules using THz absorption spectroscopy

(76) Inventors: Robert R. Alfano, Bronx, NY (US); Baolong Yu, Bronx, NY (US); Yuanlong Yang, Shanghai (CN)

Correspondence Address: DILWORTH & BARRESE, LLP, 333 EARLE OVINGTON BLVD., SUITE 702, UNIONDALE, NY, 11553, US

(21) Appl. No.: 10/827,974

(22) Filed: Apr. 19, 2004

Related U.S. Application Data
(60) Provisional application No. 60/463,354, filed on Apr. 17, 2003.

(51) Int. Cl. C12Q 1/68; G01N 21/00

(52) U.S. Cl. 435/6; 436/164

(57) ABSTRACT

A method and apparatus of detecting biological molecules, the method including the steps of: performing Terahertz (THz) absorption spectroscopy, performed in a first frequency range of 0.2 to 2.2 THz (10-79.2 cm−1), on at least one sample including a substance comprising the biological molecules, the substance being selected from at least one of tryptophan, albumin bovine, DNA, nucleotides, bacillus subtilis, spore, and DPA; calculating a frequency-dependent absorption value of biological molecules; performing THz absorption spectroscopy on at least one reference substance; detecting the substance through the frequency-dependent absorption value by comparison of absorption peaks; and outputting information proving existence of the substance in the sample. The method further creates a library of known THz frequency modes on spectra to identify the presence of unknown substance in biological and chemical composite media.

### CROSS REFERENCE TO RELATED APPLICATIONS

This application is related to U.S. Provisional Patent Application Ser. No. 60/463,354 filed Apr. 17, 2004, the contents of which are incorporated herein by reference.

### BACKGROUND OF THE INVENTION

1. Field of the Invention

The present invention relates generally to Terahertz (THz) absorption spectroscopy and more specifically to a simple, cost effective, high-performance method of detection of biological molecules using THz absorption spectroscopy.

2. Description of Related Art

Low frequency collective vibration modes of biological molecules in proteins, DNA, virus, and bacteria can provide information about type of bio-molecules present in these substances and their conformational state. Low frequency collective vibration modes are associated with collective motion of the subunits of molecules moving with respect to one another or coherent movement of a portion of a structural subunit. Similar motions are associated with conformational movements that occur during ligand binding and are critical to protein function, folding, isomerization and riboswitches.

Terahertz (THz) spectroscopy offers a new tool to probe for the presence of biological molecules in an area. As described in A. G. Markelz, A. Roitherg and E. J. Heilweil, “Pulsed Terahertz Spectroscopy Of DNA, Bovine Serum Albumin And Collagen Between 0.1 And 2.0 THz.” Chem. Phys. Lett., (2000) 320, 42-48, which is incorporated herein by reference, (hereinafter “Markelz”), E. W. Prohofsky and collaborators have predicated the helix, base twisting, and librational modes of DNA in the 20-100 cm⁻¹ range.

As described in R. Nossal and H. Lecar, “Molecular And Cell Biophysics”, 1^{st} edition (1991) Addison-Wesley, Redwood City, Calif.; W. Zhuang, Y. Feng and E. W. Prohofsky, “Predication Of Modes With Dominant Base Roll And Propeller Twist In B-DNA Poly (dA)-Poly (dT)” (1990) Phys. Rev. A41 7020-7023; and L. Young, V. V. Prabhu, E. W. Prohofsky, “Calculation Of Temperature Dependence Of Interbase Breathing Motion Of A Guanine-Cytosine DNA Double Helix With Adenima Thymine Insert” (1991) Phys. Rev. A41, 1049-1053, all of which are incorporated herein by reference, proteins are close-packed structures where changes in the arrangement of subunits in the protein take place due to photo-isomerization and enzyme actions on a sample. A conformational transition from one structure to another involves these collective modes.

As discussed in Austin, R. H., Hong, M. K., Moser, C., and J. Plombon. “Far-Infrared Perturbation Of Electron Tunneling In Reaction Centers.” (1991) Chem. Phys. 158: 473-486, which is incorporated herein by reference, molecules excited up the vibrational ladder can cross the transitional energy barrier. The dynamics of the collective modes generally occur via anharmonic interactions with other normal molecular modes leading to energy exchange. According to Austin, R., Roberson, M., and P. Mansky, “Far-Infrared Perturbation Of Reaction Rates In Myoglobin At Low Temperature.” (1989) Phys. Rev. Lett., 62: 1912-1915 and Xie, A. Meer, Alexander F. G Van Der, and Robert H. Austin. “Excited-State Lifetimes Of Far-Infrared Collective Modes In Proteins.” (2002) Phys. Rev. Lett. 88: 018102-4, which are incorporated herein by reference, it is believed that the low-frequency collective modes are responsible for the directed flow of conformational energy for a variety of biological processes ranging from primary photoisomerization events of vision to enzyme action. The motions of molecular sub-units within proteins are associated with different functions. These processes involve well-defined torsional modes along one of the C═C bonds of the polyene chain. The THz region offers a way to detect these biological molecules like in the visible, UV, and infrared region.

Far-infrared (FIR), in the range from 10μ to 1000μ, studies of materials have been limited due to weak sources and low signal to noise ratios, especially below 100 cm⁻¹. Pulsed THz time-domain spectroscopy (TDS) can be used to overcome these difficulties and have become a versatile tool for spectroscopy in FIR. Both picosecond (ps) and femtosecond (fs) time probes as well as THz frequency can be used. The THz technique has been applied to examine motion of DNA and other bio-molecules (see Markelz) for studies, not detection of the DNA.

What is needed is a method for separating molecules along the main THz absorption lines of biological molecules, where THz can be used as fingerprints to distinguish different bio-molecules and detect the presence of these bio-molecules, such as bacteria and virus, in a given area using infrared radiation.

### BRIEF DESCRIPTION OF THE DRAWINGS

The foregoing and other objects, aspects, and advantages of the present invention will be better understood from the following detailed description of preferred embodiments of the invention with reference to the accompanying drawings that include the following:

FIG. 1 is a schematic diagram of a Terahertz time-domain spectrometer;

FIG. 2 is a graph of measured Terahertz temporal profiles for a polyethylene substrate alone and a graph of measured Terahertz temporal profiles for a tryptophan film on a polyethylene substrate;

FIG. 3 is a graph of power spectra of the polyethylene substrate alone and the power spectra of the tryptophan film on the polyethylene substrate illustrated in FIG. 2, in which the insert is a graph showing logarithmic dependence of the power spectra on frequency (v);

FIG. 4 is a graph of absorbance of the tryptophan film, in which the dash line indicates a fit to the absorption data according to a sum of six Lorentzian oscillators;

FIG. 5 is a graph of the Terahertz absorption spectrum of bacillus subtilis;

FIG. 6 is a graph of the Terahertz absorption spectrum of spore; and

FIG. 7 is a graph of the Terahertz absorption spectrum of albumin bovine.

### SUMMARY OF THE INVENTION

The invention describes a method of detecting biological molecules, comprising the steps of: performing Terahertz (THz) absorption spectroscopy, performed in a first frequency range of 0.2 to 30 THz (10-79.2 cm⁻¹), on at least one sample including a substance comprising the biological molecules, the substance being selected from at least one of tryptophan, albumin bovine, DNA, RNA, nucleotides, bases, bacillus subtilis, spore, and dipicolinic acid (DPA), viruses, proteins, amino acids; calculating a frequency-dependent absorption value of biological molecules; performing THz absorption spectroscopy on at least one reference substance; detecting the substance through the frequency-dependent absorption value by comparison of absorption peaks; and outputting information proving existence of the substance in the sample. According to the present invention, a library of modes of biological and chemical substances and molecules in THz range frequency is developed by collecting absorption specters of all substances to be tested.

The presence of the biological molecules in the substance of the at least one sample is detected by comparison of electrical signal specters of the sample and the reference, where there is the presence of pre-determined absorption lines in the electrical signal specter of the sample. The electrical signals are created by conversion from optical signals in a balance detector. The converted electrical signals are intense and have a signal to noise ratio of 5000 to 1 over a large THz bandwidth.

The described absorption spectroscopy is frequency-dependent and is obtained from a mode-locked Ti:Sapphire amplifier system providing pulses greater than 90-fs, for example 200-fs pulses at a wavelength of 800 nm, with a repetition rate of 250 kHz. The amplifier system produces a strong THz pulse radiation by using optical rectification in a nonlinear medium, e.g., a ZnTe Crystal via χ⁽²⁾. Other lasers can be used, for example a Cr⁴⁺ Forsterite laser operating in 1150 nm-1300 nm and a Cr⁴⁺ YAG laser operating in 1300 nm-1600 nm range to produce THz radiation for developing a spectrometer unit using optical rectification and/or optical switching.

The first frequency range covers collective vibrational and torsional modes occurring in the sample substance to measure absorption peaks. The low frequency of the first frequency range is responsible for directed flow of conformational energy for a variety of biological motions. A second frequency range covers torsional modes along one of the C═C bands of the chain and other groups including C—H, C—N, H—O, D-O, CH₃, C—S, CH₂, CO, OO, and HO.

The invention describes measuring the absorption spectrum of biological-molecules, such as tryptophan, albumin bovine, bacillus subtilis, spore, and DPA, in the range from 0.2 to 2.2 THz (10-79.2 cm⁻¹). The THz absorption lines are used as characteristics to distinguish different biological-molecules, such as bacteria and viruses, and also to detect the existence of the biological-molecules, visual pigments, photosynthesis molecules, bacteriochlorophyll and bacteriorhodopsin. The visual pigments may include rhodopsin, photosysthesis molecules, and chromophores such as bacteriorhodopsin and bacteriochlorophyl.

### DETAILED DESCRIPTION OF THE INVENTION

The invention describes a method of using Terahertz (THz) spectroscopy for detecting biological molecules in a substance, such as bacteria. The THz absorption spectra of biological molecules, such as tryptophan, albumin bovine and bacteria, e.g., bacillus subtilis, spore, and dipicolinic acid (DPA) have been found in the range from 0.2 to 3 THz (10-99 cm⁻¹). Different absorption lines were found for different biological molecules. These THz absorption lines caused by the torsional and rotational motion of molecules, can be used to distinguish biological molecules.

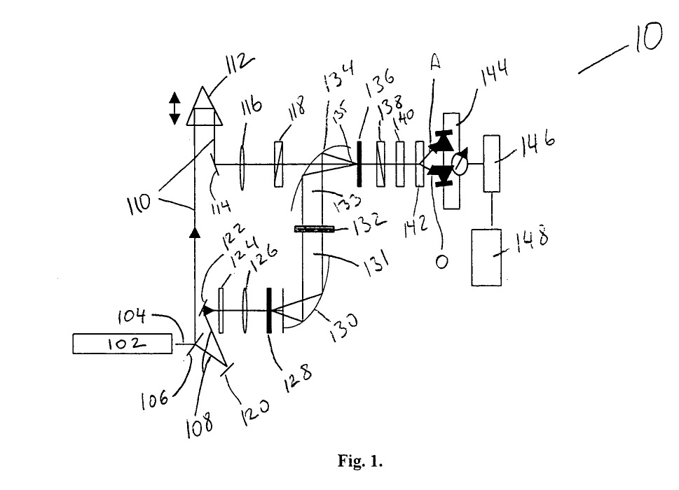

A THz time-domain spectroscopy (TDS) system 10 for measurement collection is shown in FIG. 1. A mode-locked Titanium-sapphire or Ti:sapphire laser (“Light Amplification by Stimulated Emission of Radiation”) 102 emits near-infrared light (light is a form of electromagnetic radiation). Infrared radiation is electromagnetic radiation of a wavelength longer than visible light, but shorter than microwave radiation. The Ti:sapphire laser 102 is tunable in the range from 750 nm to 1,100 nm. Titanium-sapphire refers to a lasing medium, a crystal of sapphire (Al₂O₃) that is doped with titanium ions. Ti:sapphire lasers operate most effectively at a wavelength of 800 nm. Ti:sapphire amplifier system 102 provides 200-fs pulses at a wavelength of 800 nm with a repetition rate of 250 kHz. Femtosecond (fs) is a very small unit of time equal to one million billionth of a second, e.g., 1 fs=10⁻¹⁵ s.

A biological and chemical sample 132 is positioned between an emitter crystal 128 and a detector crystal 136. The THz-TDS system 10, makes and collects the measurements of the THz absorption spectra of biological and chemical sample 132; these measurements are made by THz time-domain spectroscopy at room temperature. The THz-TDS system 10 is enclosed in dry nitrogen purged boxes (not shown) to diminish the Tera absorption due to ambient humidity. Because the THz possesses superior penetration over other materials, the sample 132 is deposited on a cell made of polyethylene substrate (not shown). The thickness of the polyethylene substrate is selected to be at least 4 mm to avoid interference from multiple reflections from the two layers of the cell substrate.

The Ti:sapphire laser 102 sends a beam of light 104 to a wedge beam splitter 106, which splits the light beam 104 in to a main beam 108, including up to 90% of the original light beam, and a control beam 110. Using pulses 108 and 110 of different optical duration ranging from a picosecond (ps), which is a very small unit of time equal to one trillionth i.e., one million millionth, of a second, e.g., 1 ps=10⁻¹² s, to fs described above, can produce Far-Infrared (FIR) radiation in χ⁽²⁾ material. For all of the components of the original light beam 104 to reach a zinc tellurium (ZnTe) detector crystal 136 at the same time, the control beam 110 is time delayed by being directed to a time delay prism 112. Using a mirror 114, the control beam 110 is directed through a lens 116 and a polarizer 118 to meet up with the main beam 108 reaching a parabolic mirror 134.

The path of the main beam 108 is redirected by mirrors 120 and 122, which are positioned in a manner as to allow the direction of the main beam 108 to be parallel to the direction of the original light beam 104. It is understood by those skilled in the art that the path of the beams described with reference to FIG. 1 is for illustrative purposes only. Any path of the beams 104, 108, and 110, leading to results described herein below is acceptable.

After path correction performed by the mirrors 120 and 122, the main beam 108 passes through a beam chopper 124, where the beam is modulated, a lens 126, and a ZnTe emitter crystal 128. Transition through the ZnTe crystal 128 produces THz radiation by optical rectification in a nonlinear medium, namely ZnTe via χ⁽²⁾ material. The electric field of the THz pulses 131 is reflected by a parabolic mirror 130 and passes through a sample of material 132 for which a specter is being graphed. After passing through the sample material 132, the electric field of the THz pulses 133 is collected by a parabolic mirror 134 and is united with the control beam 110 to result in a collected beam 135.

The collected beam 135 is detected in a second ZnTe crystal 136 via electro-optic sampling described below. The collected beam 135 passes through a cross polarizer 138, a quarter wave plate 140, and finally a Wollaston prism 142. The Wollaston prism consists of two orthogonal prisms, whose optical axes lie perpendicularly to each other and perpendicular to the direction of propagation of the incident light, in the present example collected beam 135. Light striking the surface of incidence at right angles is refracted in the first prism into an ordinary (O) ray and an extraordinary (A) ray.

A balanced detector 144 detects both rays and performs the optical rectification in a nonlinear medium and the electro-optic sampling, which is discussed in Wu, Q., Litz, M., and X. C. Zhang, “Broadband Detection Capability of ZnTe Electro-Optic Field Detectors.” (1996), Appl. Phys. Lett., 68: 2924-2926, incorporated herein by reference, (hereinafter referred to as “Wu”) and Yu, B. L., and R. Alfano. “Probing Dielectric Relaxation Properties of Liquid CS2 With Terahertz Spectroscopy. (2003) Appl. Phys. Lett. (to be published), incorporated herein by reference, (hereinafter referred to as “Yu”).

The electrical signal measurements, converted from the optical by the balanced detector 144, are measured by a lock-in device 146 and are stored and displayed on a computing device 148 having a video and audio display, a printer, and networking capabilities (not shown). The computing device may make audio announcements, e.g., via a speaker, and transmit the analyzed findings to other computing devices via a network, for example the Internet.

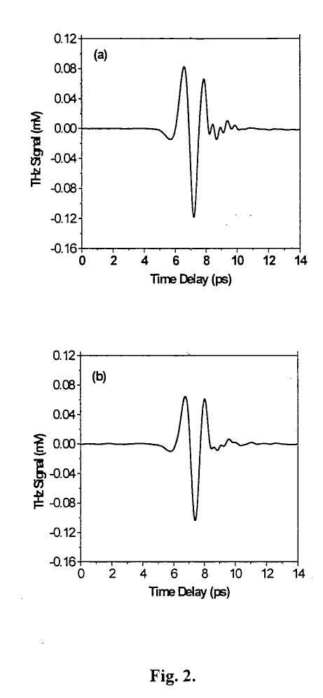

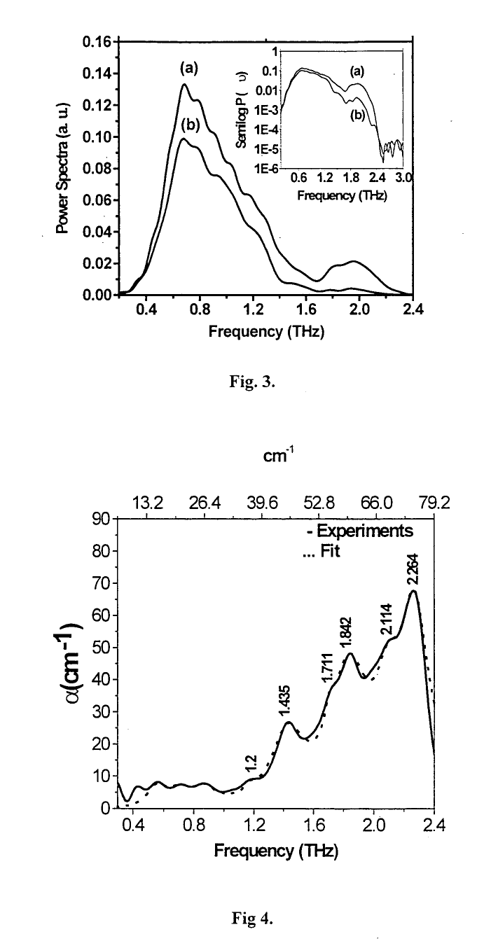

FIG. 2 illustrates the electrical signal measurement specters in a graph (a), a reference graph of the THz temporal profiles after transmission through an empty polyethylene cell, and in a graph (b), the graph of THz profiles after transmission through a tryptophan film. FIG. 3 shows power curves marked with letters (a) and (b) respectively resulting from performance of a Fourier Transform of the temporal profiles of graphs of (a) and (b) of FIG. 2 for both the substrate and the deposition of tryptophan on the substrate. The frequency-dependent absorption of the sample 132 can be determined by performing the following calculation: ln(Pₛₐₘₚₗₑ/P_{reference}). The absorption peaks of tryptophan in the THz region from 0.2 to 2.2 THz is shown in FIG. 4.

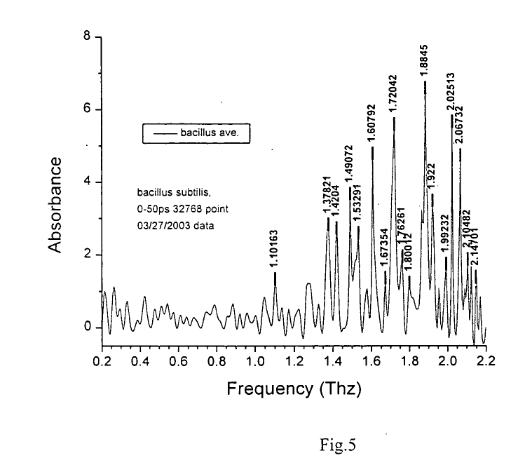

In another example, shown in FIG. 5, the THz frequency-dependent absorption of bacillus subtilis (used as sample 132) in the THz frequency region is shown. In the shown spectrum, some water vapor absorption lines such as: 1.09, 1.41, 1.60, 1.71 THz are found. Other lines, such as 1.38, 1.49 1.53, 1.88 THz are found characteristic of bacteria.

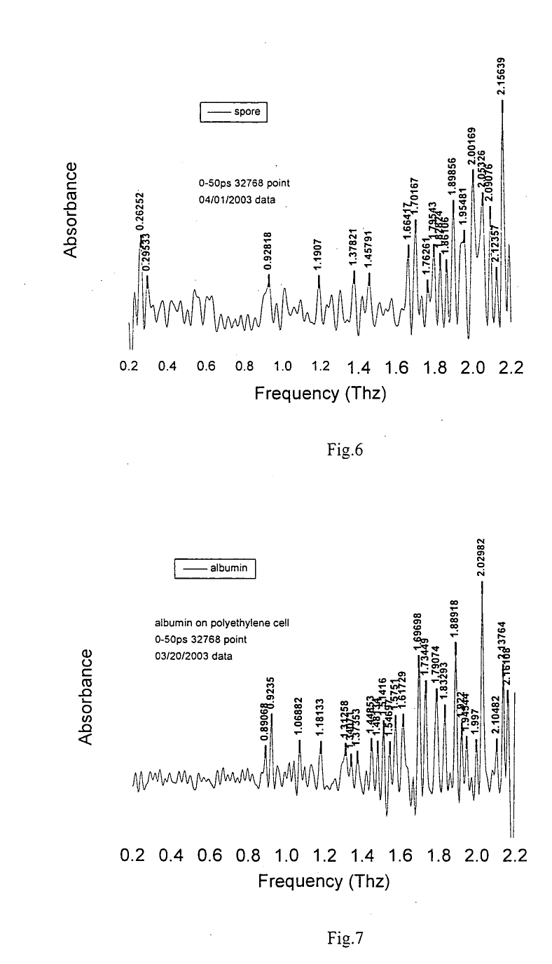

FIG. 6 illustrates a frequency-dependent absorption of bacteria spore (used as sample 132) in the THz frequency region. In the spectrum, some lines are the same as those of bacillus subtilis of FIG. 5 while others are different, indicating distinct characteristics.

FIG. 7 illustrates a frequency-dependent absorption of protein albumin bovine (used as sample 132) in the THz frequency region. As can be seen, distinct characteristic lines, except for vapor lines, are seen in the spectrum.

The frequency-dependent main THz absorption peaks of specific bio-molecules: L-tryptophan, protein, albumin bovine, DNA, e.g., salmon tests, nucleotide, bacteria, e.g., bacillus subtilis, spore, and dipicolinic acid (DPA) in the range of 0.2 to 2.2 THz are summarized in Table 1 below. As can be seen from the Table, the absorption peaks for different bio-molecules are different. These differences can be used as fingerprints to distinguish bio-molecules. These exact frequencies can change depending on the environment that these substances are located in and surrounded by due to polar and nonpolar environments and pH.

TABLE 1

| | Bacteria | | DPA |
|---|---|---|---|
| L-Tryptophan | (bacillus | DNA (salmon tests) | (Dipicolinic acid) |
| (THz) | subtilis) (THz) | (THz) | (THz) |
| | | 0.853 | |
| | 1.051 | | |
| | | 1.134 | |
| 1.200 | 1.238 | | |
| 1.435 | | 1.472 | |
| | 1.538 | | |
| | | | 1.622 |
| 1.711 | 1.725 | 1.702 | |
| 1.842 | | | 1.81 |
| | 1.924 | 1.908 | 1.997 |
| | 2.04 | | |
| 2.114 | 2.119 | 2.178 | 2.142 |
| 2.264 | 2.231 | 2.231 | |

While the invention has been shown and described with reference to certain preferred embodiments thereof, it will be understood by those skilled in the art that various changes in form and details may be made therein without departing from the spirit and scope of the invention as defined by the appended claims.

What is claimed is:

1. A method of detecting of biological molecules, the method comprising the steps of:
performing Terahertz (THz) absorption spectroscopy on at least one sample including a substance comprising the biological molecules;
calculating a frequency-dependent absorption value of biological molecules of said at least one sample;
detecting the substance through the frequency-dependent absorption value by comparison of absorption peaks; and
outputting information proving existence of the substance in the sample.

2. The method of claim 1, further comprising a step of performing THz absorption spectroscopy on at least one reference substance.

3. The method of claim 2, further comprising a step of collecting a plurality of THz absorption specters of a plurality of reference substances into a library, wherein said library is used for identification of molecules in the detecting step.

4. The method of claim 1, wherein the substance is selected from one of tryptophan, albumin bovine, bacteria, DNA, RNA, nucleotides, bases, bacillus subtilis, spore, proteins, amino acids, viruses, riboswitches, dipicolinic acid (DPA), visual pigments, genes, and enzymes,
wherein different absorption lines being found for each substance comprising biological-molecules, the absorption lines are caused by the torsional and rotational motion of molecules being used to distinguish biological molecules.

5. The method of claim 4, wherein the absorption lines from the at least one sample indicate the presence of the substance.

6. The method of claim 4, wherein a ratio of absorption lines of the at least one sample are used for distinguishing different molecules and determining the presence of the substance.

7. The method of claim 4, wherein the absorption lines from the at least one sample are compared with compounds of the absorption lines from different samples of said at least one sample for detecting the biological molecules to determine if the substance comprising the biological molecules is present.

8. The method of claim 4, wherein the presence of the biological molecules in the substance of the at least one sample is detected by comparison of electrical signal specters of the sample and the reference, where the presence of pre-determined absorption lines in the electrical signal specter of the sample.

9. The method of claim 8, further comprising a step of converting optical signals into electrical signals in a balance detector, wherein the electrical signals are intense and have a signal to noise ratio of 5000 to 1 over a large THz bandwidth.

10. The method of claim 4, wherein the visual pigments comprise rhodopsin, photosysthesis molecules, and chromophores selected from bacteriorhodopsin and bacteriochlorophyl.

11. The method of claim 1, wherein the THz absorption spectroscopy is performed in a first frequency range of 0.2 to 2.2 THz (10-79.2 cm⁻¹).

12. The method of claim 11, wherein the first frequency range covers collective vibrational and torsional modes occurring in the at least one sample substance to measure absorption peaks.

13. The method of claim 12, wherein low frequency of the first frequency range is responsible for a directed flow of conformational energy for a plurality of groups of biological motions arising from torsional, rotational, and translation motion and coupling to electronic-vibrational levels.

14. The method of claim 12, wherein a second frequency range covers torsional modes along one of the C═C bands of the chain and a plurality of groups selected from C═C, CO, OO, HO, C—H, C—N, CH₂.

15. The method of claim 1, wherein absorption spectroscopy is frequency-dependent and is obtained from a mode-locked Ti:Sapphire amplifier system providing pulses greater than 90 fs at a wavelength in a range from 750 nm to 1,100 nm, with high repetition rate.

16. The method of claim 15, wherein the amplifier system produces a stronger THz pulse radiation by using optical rectification in a nonlinear medium.

17. The method of claim 16, wherein the nonlinear medium is a ZnTe Crystal via χ⁽²⁾.

18. The method of claim 1, wherein said step of performing Terahertz (THz) absorption spectroscopy uses spectroscopy in the range from 0.2 THz to 30 THz.

19. The method of claim 1, wherein absorption spectroscopy is obtained from an amplifier system using lasers selected from a Cr⁴⁺ Forsterite laser operating in 1150-1300 nm and a Cr⁴⁺ YAG laser operating in 1300-1600 nm range to produce THz radiation for developing a spectrometer unit using optical rectification and/or optical switching.

20. The method of claim 1, wherein absorption spectroscopy is obtained from an amplifier system using pulsed lasers in a range of 10 fs-200 fs to produce THz radiation selected from semiconductors, doped fibers, and solid state lasers.

21. The method of claim 1, further comprising the steps of:
photoexciting the sample with optical fs radiation; and
probing the torsional and rotational motion of excited molecules and changes in isomerization as a function of time delay,
wherein the dynamics of molecules in the sample are measured using a time resolved spectrometer to obtain relaxation lifetimes (τ) of the molecules in the sample to determine presence of unknown species in THz region of the spectra to yield τ at THz frequencies, lifetimes (τ) are used to determine the presence of substances in the sample.

22. The method of claim 1, wherein time-resolved spectroscopy is used in said step of performing absorption spectroscopy.

23. The method of claim 1, wherein said step of performing THz absorption spectroscopy further comprising a step of imaging said at least one sample including said substance comprising the biological molecules via a step selected from scanning of THz and moving of optical beams over an area of said at least one sample.

24. An apparatus for detecting biological molecules, the apparatus comprising:
a Terahertz (THz) absorption spectrograph for performing spectroscopy in a first frequency range of 0.2 to 2.2 THz (10-79.2 cm−1), on
at least one sample including a substance comprising the biological molecules, the substance being selected from at least one of tryptophan, albumin bovine, DNA, RNA, nucleotides, bases, bacillus subtilis, spore, proteins, amino acids, viruses, riboswitches, dipicolinic acid (DPA), and visual pigments, and on at least one reference substance; and
a computing device for
calculating a frequency-dependent absorption value of biological molecules;
detecting the substance through the frequency-dependent absorption value by comparison of absorption peaks, and
outputting information proving existence of the substance in the sample.

25. The apparatus of claim 24, wherein said THz absorption spectrograph further comprises:
a means for imaging said at least one sample including said substance comprising the biological molecules via a step selected from scanning of THz and moving of optical beams over an area of said at least one sample.
